(19) United States
(12) Patent Application Publication
(10) Pub. No.: US 2005/0203427 A1
(43) Pub. Date: Sep. 15, 2005

(54) Stenosis detection device

(75) Inventor: William V. Judy, Bradenton, FL (US)

Correspondence Address: BROWDY AND NEIMARK, P.L.L.C.;624 NINTH STREET, NW, SUITE 300, WASHINGTON, DC, 20001-5303, US

(73) Assignee: IMPEDANCE VASCULAR IMAGING PRODUCTS, LLC, Bradenton, FL (US)

(21) Appl. No.: 10/800,005

(22) Filed: Mar. 15, 2004

(51) Int. Cl. A61B 5/04; A61B 5/02

(52) U.S. Cl. 600/506

(57) ABSTRACT

The present invention provides a method and a device for determining a peak blood flow signal of a blood flow through at least a section of a selected coronary artery of a beating heart of a mammal, in particular a human being, wherein said device comprises a bioimpedance measuring device. The method and device selects part of a bioimpedance signal, and calculates a peak velocity from it. This may e.g. be used to map the peak blood flow velocity along a coronary artery, in order to find possible stenoses in the vessel.

### FIELD OF THE INVENTION

The present invention relates to a device and a method for determining a peak blood flow signal of a blood flow through at least a section of at least one selected coronary artery of a beating heart of a mammal, in particular a human being. In particular, the device and method may be used to help determine the location of stenosis in a coronary artery.

### BACKGROUND OF THE INVENTION

Stenosis is a stricture or narrowed passage in a blood vessel. This may have various causes, but often it is caused by the deposition of fatty substances on the inner wall of the blood vessel. The narrowed passage may hinder the flow of blood and hence the oxygen supply e.g. to a muscle. A very important example of stenosis is the stenosis in a coronary artery, which provides blood to the heart muscle. The treatment of coronary stenosis is a major and very costly part of health care.

Hence, many efforts have been made to develop methods and systems for the detection of coronary stenosis in as early a stage as possible. Some of the methods are invasive. For example, a catheter is brought into a coronary artery, with the help of which a blood flow velocity may be determined. This velocity has a known relationship to the relative cross sectional area of the vessel and hence stenosis thereof. In another method, coronary angiography, one injects contrast fluid, and determines an image of the vasculature with the help of radiation, e.g. X-rays. These invasive methods require the presence of a doctor, and are a risk to the patient. Hence non-invasive methods are preferred.

Most such non-invasive methods require the use of bulky and costly machines, e.g. MRI-apparatus. These methods are not suitable for wide spread use in physicians' practices, or travelling doctors etc. Fast CT-scans are a new non-invasive method, but it is limited by poor detection of soft plaque. WO-A-98/23211 discloses a method and apparatus for measuring coronary stenosis. The method uses beams of ultrasound aimed at coronaries. The Doppler-shift of beams reflected by blood in the vessels allows to locate any stenosis.

However, it is necessary to use a separate device to determine a time gate in which the heart itself is not moving, which would cause additional measuring error. Furthermore, this method only allows a 2-dimensional section to be measured, and hence necessitates active aiming and scanning across the heart, which is hardly possible to automate.

Hence there is a need for a method and device for determining a peak blood flow signal of a blood flow through at least a section of at least one selected coronary artery, which method can easily be automated.

### SUMMARY OF THE INVENTION

An object of the present invention is to provide a device and a method for determining a signal indicative of a peak blood flow signal of a blood flow through a coronary artery, which do not show the above disadvantage.

Thereto the device is characterized in that it comprises a bioimpedance measuring device, which at least comprises
at least two measuring electrodes,
an impedance measuring device for measuring an impedance signal Z between a pair of the measuring electrodes, and
processing means, which are able to determine a first time-derivative dZ/dt for the measured impedance signal Z, and to separate from the first time-derivative dZ/dt a peak signal PS that occurs first after the beginning of diastole of the heart during a heart beat,
   and wherein the processing means are able to determine a maximum-value MAX of the peak signal PS.

Thus a relatively simple device is provided, which may be used without supervision by a physician, and the function of which which may be easily automated.

Generally, the processing means comprise some kind of computer or logical circuit, designed to convert the measured impedance signal Z into a first time-derivative dZ/dt. This may be performed on the analogue signal or on a sampled and digitized signal. The determination of the first time-derivative is a well known method for the person skilled in the art, and standard devices and/or computer programs are available for the performance of this task. Furthermore, the processing means are able to separate from a first time-derivative dZ/dt a peak signal PS that occurs first after the beginning of diastole of the heart during a heart beat.

It was found that the filling of coronary arteries takes place during early diastole, i.e. in the first part of the phase of the heart beat in which the heart muscle relaxes. In fact, diastole begins with closing of the aortic valve. The beginning of diastole may be determined in any way known in the art. Preferably, it is determined from the dZ/dt signal, in that diastole begins at the moment where dZ/dt assumes a minimum value following a maximum value. This will be elucidated with reference to the drawings.

The first signal to follow the beginning of diastole, when measuring an impedance signal across the heart has been found to be a peak indicating the filling of either a left or a right coronary artery, depending on which part of the heart is being measured. It turned out that blood flow signals from coronary arteries could be measured as long as these arteries did not enter the heart muscle. Furthermore, in addition to the separation in time between the left and right coronary arteries, it was found that signals from vessels on the anterior surface of the heart are separated from signals from vessels on the posterior surface, because of the presence therebetween of heart muscle and the cardiac chambers. Hence, in view of the device according to the invention, the separating of the peak signal PS may simply be performed by gating the measuring in a time slot that starts with the beginning of diastole. The time slot may be taken to be at least as long as the isovolumetric relaxation time of the heart. This is the initial portion of the period of time during relaxation of the heart muscle and during which all valves are closed and hence no blood enters or leaves the heart ventricles. Normally this period is about 0.5 to 0.75 seconds. However, any other way to separate the desired peak signal may be used as well. It could be contemplated to use signal recognition means, to analyze the Z-signal or dZ/dt-signal pattern to isolate the characteristic peak.

The processing means are further able to determine a maximum value MAX of the peak signal PS thus separated. A maximization device is a standard tool for a person skilled in the art. It can be embodied e.g. as a computer program to process digitized data, or as an anologue circuit embodied as a “max hold”-circuit. The value of MAX thus found is indicative of the peak blood flow velocity in the measured section of the coronary artery. Even though the distance between the measuring electrodes plays a role in determining the peak blood flow value, general information may be obtained from the value of MAX. Any complete obstruction of a vessel, for example, would show up as a zero signal.

With the above device it is possible to scan along the surface of the chest, in order to map one or more coronary arteries. Thereto, the pair of measuring electrodes is moved along the chest surface, each time taking a new signal. This is fine when only a few spots need to be measured, e.g. when the measurement relates to the check of a particular section of one particular coronary artery. Preferably, however, the bioimpedance measuring device comprises a plurality of measuring electrodes. With a plurality of measuring electrodes, it is much easier and quicker to scan a certain area of the heart. This decreases the fiddling with the patient. Also, it improves the quality of the measurement, in that the heart function will vary less in a shorter time.

The plurality of measuring electrodes may be a number of separate electrodes to be applied separately to the body of the patient. However, this would be a very laborious and complex device, because every electrode must be applied separately. This device can not be used with ease in e.g. an emergency situation. Furthermore mistakes are apt to be made since it is difficult to keep the overview over all these separate electrodes. In a preferred embodiment, the plurality of measuring electrodes is arranged in a mesh. Here, the electrodes may be provided as mutually electrically insulated spot electrodes in a flexible sheet of material, or as a network etc. This mesh or regular pattern of measuring electrodes allows a well structured measurement, and easy reference to later measurements. The mesh may be a rectangular mesh, with rows and columns of measuring electrodes. However, some other pattern may also be used.

Expediently, all neighbouring measuring electrodes are substantially equidistant. This most regular pattern allows for the easiest reference between electrodes and measured location, and for greatest flexibility. If desired, an oblong mesh may be used as well.

Preferably, the processing means are able to determine a peak blood flow signal PF for said peak signal PS, by dividing said maximum value MAX by the distance between those two of said at least two measuring electrodes between which the impedance signal Z was measured, when the device is being used. This is standard arithmetic and may be embodied in any computer program, logical or analogue circuit known in the art. Since the impedance signal, and hence the peak signal PS, depends on the amount of tissue between the measuring electrodes, hence on the length between the measuring electrodes, some gauging is desirable to be able to compare measurements. Thereto the measured peak signal PS is divided by the distance between the measuring electrodes. This distance correction may be used when using a device with only two electrodes, which are moved across the chest surface. Furthermore, the correction may be used with the mesh of measuring electrodes, when the distance between neighbouring electrodes is not regular. Also, it may be used when the mesh distance is regular, but a pair is selected which does not consist of two neighbouring electrodes. However, it is stressed that, as long as the distance between the used measuring electrodes remains a constant, it is not necessary to divide the signals by said distance. The distance is automatically accounted for when a calibration of the method or device is performed, to correlate a measured signal to a blood flow value. This should be done at least when the method or device is used for the first time, although this may be done in the factory. In this calibration other factors are included, such as the sensitivity of any part used.

With the results of the measurements, the peak blood flow signal between the selected pair of measuring electrodes is determined by means of the processing means. With the help of this peak blood flow signal information may be obtained about the blood flow in the selected coronary artery. This will be elucidated in the following.

In an advantageous embodiment of the device according to the invention, the processing means further comprise scanning means, which are able to select two or more pairs of the plurality of measuring electrodes during the heart beat, the processing means for each of the pairs being able to determine the peak blood flow signal PF in that section of the selected coronary artery which is bounded by the pair of measuring electrodes selected by the scanning means. Thus, the device may be provided with scanning means that enable the user of the device to select any two electrodes for measuring a peak signal PS therebetween. They may be embodied as a kind of switch board, allowing the user to select the desired measuring electrodes by selecting the corresponding buttons or switches on the board.

Otherwise, the scanning means may comprise e.g. a computer, by means of which the selection may be performed automatically, after entering the desired command for measuring. This way it is very easy to scan consecutively every neigbouring pair in the mesh of measuring electrodes. Such pairs may be selected between two neigbouring rows or columns.

If the impedance measuring means have only one input channel, then, when measuring in an analogue way, only one peak signal PS may be determined in one heart beat. In order to determine change of the peak blood flow signal along a coronary artery, it is necessary to measure at various locations, i.e. various pairs of electrodes. Hence in a setup with only one analogue input, measurements during various heart beats are necessary.

Depending on the number of input channels of the measuring device it is possible to measure more than one pair of electrodes at the same time, i.e. during the same heart beat. This holds for an analogue way of measuring. By thus reducing the number of required heart beats, there will be less variations in the heart function during the measurements, which would improve the accuracy.

However, if a sampling way of measuring is adopted, it is also possible to measure more than one pair during the same heart beat, but without the need for more than one input channel. The impedance measuring means and the scanning means may cooperate to sample for a first time a first signal from a first selected pair. Then a first signal from a second selected pair, e.g. a neigbouring pair, may be sampled for a first time, and so on, until every desired pair has been sampled a first time. Next, the sampling procedure is repeated with every desired and selected pair, to obtain a second signal for every pair, and so on, until enough sampled signals have been obtained for every desired pair of electrodes, such that a complete peak signal PS may be derived, and processed by means of the processing means.

This way it is possible, of course depending on the speed of sampling and measuring of the scanning means, the impedance measuring means respectively, to measure e.g. all electrode pairs in two neighbouring rows. If the impedance measuring means additionally have more than one input channel, advantageously every pair of neigbouring rows may be sampled during the same heart beat, i.e. in one beat of the heart a complete scan of the peak blood flow in the coronary artery or arteries is possible. Hence in a preferred embodiment of the device according to the invention, the scanning means are able to select all pairs of neighbouring measuring electrodes during one heart beat.

Measured and processed signals may be further processed and evaluated by hand. Alternatively, it is possible to display the values of the peak blood flow signals along the artery. Expediently, a preferred embodiment of the device according to the invention further comprises display means for representing said peak blood flow signal PF as a function of position along said at least one selected coronary artery. Such display means may be realized in any way known in the art. For example, the distance along the selected coronary artery may be represented on a horizontal axis, whereas the value of the measured peak blood flow signal may be represented on a vertical axis. Alternatively, a graph may represent the selected coronary artery, along which the measured peak blood flow signal may be represented as e.g. a color, different colors corresponding with different values. With the help of such a device it becomes possible to obtain an overview of the peak blood flow velocity along the length of the blood vessel. For peak blood flow through a certain part of a blood vessel equals the volume of the blood that passes through that part of the vessel times the velocity of that blood. Since the volume of the blood does not change (appreciably) as long as no side vessel branches off, the peak blood flow signal represents the peak velocity signal. E.g. for the major coronary arteries this peak signal will rise steadily as a function of distance to the origin of the vessel. Since the cross-section of these vessels decreases steadily as a function of the distance from the origin of the vessel, the peak velocity will increase correspondingly. This will be elucidated below. With this overview of the peak blood flow velocity, e.g. any stenosis in a coronary artery may be indicated relatively easily.

In a preferred embodiment of the device according to the invention, the plurality of measuring electrodes can substantially cover the heart. With this embodiment a complete view of the peak blood flow signal in the major surface vasculature of the heart may be obtained. It is however also possible that the mesh of measuring electrodes covers only part of the heart, e.g. one half of one side of the heart, corresponding to the area of the heart where a major coronary artery runs.

Furthermore, the invention relates to a method for determining a peak blood flow signal PF of a blood flow through at least a section of at least one selected coronary artery of a beating heart of a mammal, in particular a human being, comprising the steps of
applying a bioimpedance measuring device at least comprising impedance measuring means and at least two mutually spaced measuring electrodes to the body of the mammal, wherein at least the section of the at least one selected coronary artery is bounded by a pair of measuring electrodes of the at least two measuring electrodes,
measuring an impedance signal Z between the pair of measuring electrodes, by means of the bioimpedance measuring device, which signal Z depends on the blood flow through that section of the at least one selected coronary artery which is bounded by the pair of measuring electrodes,
determining a first time-derivative dZ/dt of the impedance signal Z.
separating from the first time-derivative dZ/dt a peak signal PS that occurs first after the beginning of diastole of the heart during a heart beat,
determining for the peak signal PS a maximum value MAX of the peak signal PS.

With this method it is possible to obtain a signal which is indicative of the peak blood flow velocity in the selected artery. However, it should be stressed that the distance between measuring electrodes influences the measured signal. Nevertheless, for example an occlusion in a selected coronary artery may be easily determined, because suddenly a much smaller or no signal is measured.

Again, as discussed earlier, the separation may be realized by a gating procedure, i.e. by only measuring during a certain period of time. This period of time is early diastole, during some 0.50 to 0.75 seconds. Because of the measured signals being relatively superficial signals, which hardly interfere with signals from neighbouring coronary arteries, in most cases only one of two possible peaks will be measured. Signals from arteries of the left coronary system show up slightly earlier than signals from arteries of the right coronary system. Hence no further separating between these two peaks is necessary. This will be further elucidated with reference to the drawings.

In an advantageous embodiment of the method according to the invention, a plurality of impedance signals Z are determined along a plurality of locations along the at least one coronary artery, wherein for each of the impedance signals Z a first time-derivative dZ/dt is determined, a peak signal PS is separated therefrom, and a maximum value MAX is determined from the peak signal PS. By thus obtaining a number of measurements, information may be obtained about various sections of one or more coronary arteries. Some profile of the measured values may be used to determine the condition of the blood vessels.

In a preferred embodiment of the method according to the invention, a peak blood flow signal PF is determined for the peak signal PS by dividing the maximum value MAX of that peak signal PS by a distance between the electrodes between which that peak signal PS was determined. This distance correction has been elucidated with respect to the device according to the invention. This correction may be used to compare signals between different locations along the artery, but also between different moments in time for the same location along the artery.

Various experiments have indicated that the obtained signal PF shows approximately a one-to-one correspondence with the actual peak blood flow velocity as measured e.g. with a catheter. Even if some numerical factor should be used, it is always a substantially linear relationship. Hence the relative variation of the actual peak blood flow velocity may be determined with the method according to the invention. Hence increases and decreases in the peak blood flow velocity in an artery may be determined.

It is to be noted that it is desirable to measure peak blood flow velocities, or signals indicative thereof, instead of peak blood flows. Even when a blood vessel is narrowed or constricted by up to 80%, the blood flow volume itself need not be diminished. This holds for a resting person. Of course, if by physical exercise etc., the basic blood flow increases, the blood can no longer pass unhindered through the constricted vessel, and symptoms of stenosis will become noticeable in the blood flow volume. However, this is not beneficial to the patient. The present method does away with the necessity for exercise to establish stenosis, because even in a minor constriction, the local blood flow velocity will increase rather sharply.

Although it is possible to use only two measuring electrodes to obtain all the necessary information, in a preferred embodiment a plurality of measuring electrodes is applied to the body. This greatly improves the flexibility and the accuracy of the body, as has already been explained.

Preferably, neighbouring measuring electrodes are applied at substantially equal distances. By taking substantially equal distances between measuring electrodes, the operation of dividing by the distance between the electrodes is much simplified, even to the extent that it is no longer necessary to do so. The term “substantially equal” means that the relative variation in mutual distance is at most equal to the allowed or desired relative error margin.

In a preferred embodiment, the method according to the invention further comprises the use of scanning means which are able to select at least two pairs of measuring electrodes. Though theoretically it would be possible to use only two measuring electrodes and scan the desired area, by using more electrodes and scanning means to select any desired pair of these electrodes, the procedure is much simplified. It is contemplated that various pairs may simply be selected by pushing the appropriate or corresponding buttons on a switch borad, or by indicating them in a computer program. It should be noted that it is possible to use one measuring electrodes for more than one pair, so a minimum of three measuring electrodes is required for this embodiment.

An advantageous embodiment of the method according to the present invention further comprises the step of representing the peak blood flow signal PF as a function of position along the at least one selected coronary artery. This way a suitable representation of the measured data becomes possible. As described hereinbefore, it is for example advantageous to plot the value of PF to see where in the artery a constriction, a dilation etc. is present.

An expedient embodiment of the method further comprises the step of graphically highlighting those sections along said at least one selected coronary artery in which an increase in said peak blood flow signal PF is followed by a decrease in said peak blood flow signal PF, as seen in the direction of said blood flow. This offers a quick and efficient way of indicating possible locations of stenosis. The highlighting may e.g. be realized as a color which changes with the local value of PF, or only locations where a sudden drop of the relative value of PF is measured, say 25% or 50% over a few millimeters.

In a preferred method according to the invention, the graphically highlighted parts are indicated on a model of a surface vasculature of the heart. With the help of this method, it becomes very easy to visualize the location of any problems with the coronary vasculature. This not only helps the physician with the explanation thereof to the patient, but also to visualize the location when e.g. surgery is needed.

In a preferred embodiment of the method according to the invention, substantially all major surface coronary arteries of the heart are selected. Even when only one artery is in a serious or critical condition, it is preferable to study all arteries in one step, or one series of measurements. They may be selected simultaneously or consecutively, depending on the possiblities of the measuring and/or scanning device.

In an advantageous method according to the invention, the plurality of measuring electrodes covers the heart substantially completely. It then becomes possible to cover and measure the heart in one step, or at least a drastically reduced number of steps, instead of applying the electrodes to measure one region of the heart, and thereafter the next, and so on. This greatly reduces fiddling with the patient, which also helps to reduce stress in the patient, and hence variations in the blood flow.

These and other advantages of the invention will now be further elucidated with reference to the drawings.

### BRIEF DESCRIPTION OF THE DRAWINGS

FIG. 1 shows a diagrammatic representation of an embodiment of the device according to the present invention, as applied to a human body.

FIGS. 2a-d show measuring signals useful for understanding the method of the invention.

FIG. 3a-c show details of measuring signals to indicate signals from left and right coronary vessels.

FIG. 4a-d show peak signals separated from the signals of FIG. 3, to be used in the method according to the invention.

### DETAILED DESCRIPTION OF THE PREFERRED EMBODIMENTS

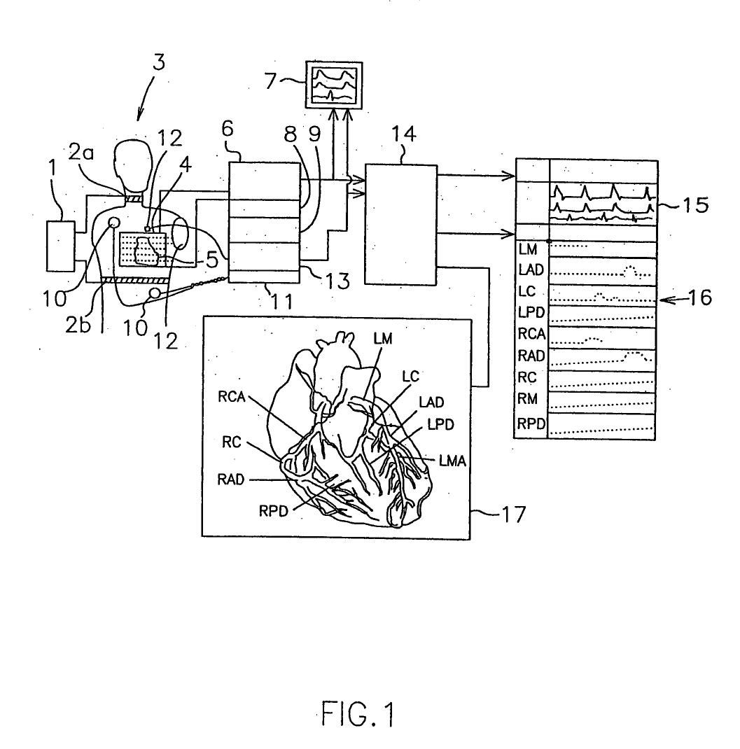

FIG. 1 shows a diagrammatic overview of the device according to the present invention, as applied to a human body 3. In FIG. 1, reference numeral 1 represents a current source, connected to supply electrodes and, which are applied to the upper part of a human body 3.

A mesh 4 of measuring electrodes is connected to an impedance measuring means 6, and to scanning means 9. The mesh 4 of measuring electrodes substantially covers the heart 5 of the human body 3. The mesh 4 is a rectangular mesh, with an interelectrode distance of 1 mm, though any other suitable distance is possible.

The impedance measuring means 6 are connected to a first monitor 7. EKG electrodes 10, which are also applied to the upper part of the human body 3, are connected to EKG measuring means 11. Phonocardiograph 12 is applied to the upper part of the body as well, and are connected to phonocardiogram measuring means 13. A correct position for the phonocardiagraph means 12 is at the 3^{rd} intercostal space just left of the sternum.

The impedance measuring means 6, scanning means 9 and EKG measuring means 11 are connected to processing means 14. In its turn, the processing means 14 are connected to a number of outputs, viz. a second monitor 15, display means 16 and heart display means 16.

In the present invention, use is made of a bioelectrical impedance measurement of the heart to determine signals which are indicative of the blood flow through a coronary artery, more specifically a surface coronary artery.

The current source 1, supply electrodes 2a,b, the mesh 4 of measuring electrodes and the impedance measuring means 6 are generally referred to as a bioimpedance measuring device. Apart from the special mesh 4 of measuring electrodes, this may basically be any bioimpedance measuring device known in the art.

Generally, in a bioelectrical measurement method an oscillating electric field is established across the region of interest, i.e. here the heart 5. Movements, and especially rapidly changing movements, of conductive fluids such as blood, cause changes in the impedance of the measured region. These changes can be detected as varying voltages across the measuring electrodes used. Nevertheless, in this application these voltages will be called impedance signals.

More specifically, the current source 1 is preferably a constant current source, which may supply a current of between 0.5 mA and 5 mA, and with a frequency of between 50 kHz and 250 kHz, for example 4 mA and 100 kHz. The supply electrodes 2a,b may be spot electrodes, strip electrodes or a combination thereof, for example strip electrodes.

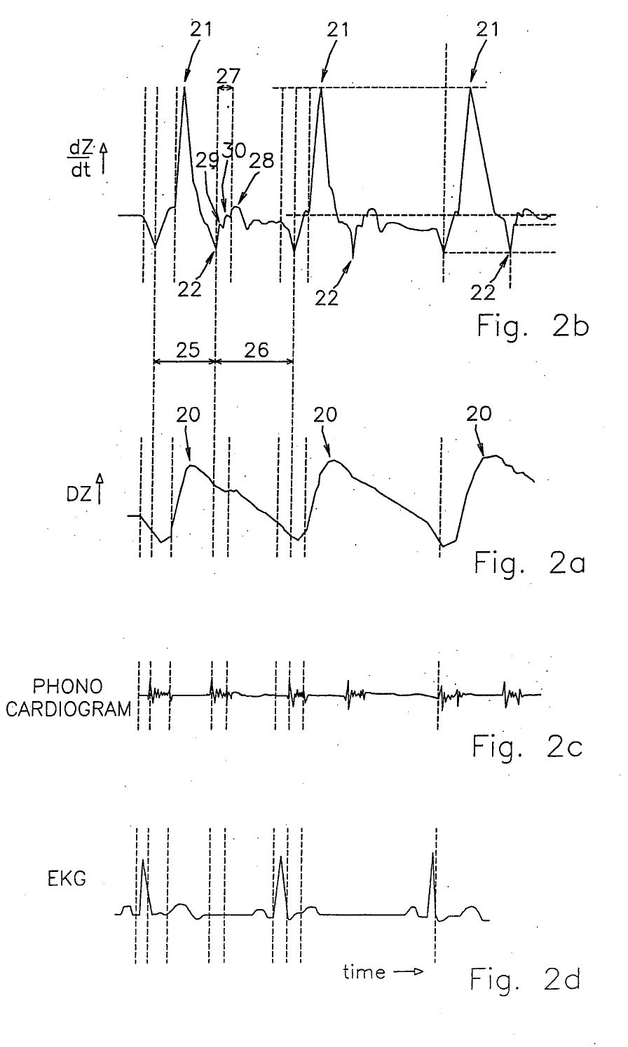

The EKG electrodes 10 and the EKG measuring means 11, as well as the phonocardiograph 12 and the phonocardiogram measuring means 13 are optional, and may provide an EKG signal, a phonocardiogram, respectively, which may be useful in evaluating and processing the measured impedance signals. This will be discussed in connection with FIG. 2a-d. The EKG electrodes 10 may be any type known in the art, and may be applied e.g. in the Lead II configuration. The phonocardiograph 12 and phonocardiogram measuring means 13 are likewise not particularly limited. The phonocardiograph 12 may be applied in its most favourable position, being the 3^{rd} intercostal space left of the sternum.

The impedance measuring means 6 may be a volt meter, oscilloscope et cetera. It only needs to measure a voltage signal. However, since this voltage depends on the impedance of the measure part of the body, it will be referred to as an impedance or impedance signal.

The impedance measuring means 6 may be connected to an optional first monitor 7. The first monitor 7 may provide a visual check of the quality of the measured impedance signal. If for example there is a bad contact, too much noise or any other influence to adversely affect the signal, this may be detected in the signal shown on the screen of the first monitor 7. This allows the correct action to be taken. It is also possible to connect other measuring instruments with the first monitor 7, for the same reason. E.g. the EKG measuring means 11 may be connected, to show the electrical activity of the heart. This is not only useful to assess the quality of the measured EKG signals, but also to be able have an extra measure of time when processing the impedance signals.

The impedance measuring means 6 may have one or more analogue inputs, and alternatively or additionally, one or more digital inputs, to measure one or more impedance signals at the same time. The impedance measuring means 6 may comprise sampling means 8. The sampling means 8 may be an electronic circuit, or a computer program to control the impedance measuring means 6 in connection with the selected electrodes. These sampling means may either sample an analogue input signal, or be used to measure a digital input signal.

The scanning means 9 are optional, though preferred. With the help of the scanning means 9 it becomes possible to measure more signals per input, during one heart beat, i.e. at the same time. The scanning means 9 thereto cooperate with sampling means 8 and the mesh 4 of measuring electrodes, such that samples are taken of every signal to be measured. Preferably a high sampling frequency is used, in order to obtain a sufficiently accurate measurement of many pairs of measuring electrodes.

The processing means 14 will process the measured signals into data indicative of the peak blood flow through parts of the coronary arteries. The method thereto will be explained hereinbelow.

The processing means 14 may be connected with an optional second monitor 15. The second monitor 15 may be used to display the sampled, and if necessary reconstructed, measured impedance signals, as a check whether the sampling procedure has worked properly. It is also possible to show the separated peak signals etc. Again, the main purpose of this monitor is visual quality control, since the method itself is not influenced by the second monitor 15.

The processing means 14 may also be connected with display means 16. By means of these display means 16, the measured peak blood flow signals may be displayed as a function of length along the selected coronary artery. Thereto a coupling has to be made between a signal measured between two specific electrodes and the length along the selected coronary artery, that corresponds with the selected pair of measuring electrodes. This may be done by hand by measuring out the distance from the origin of the coronary artery, i.e. the first pair of electrodes to give a signal for that coronary artery. It may also be done with the aid of some kind of computer program to automatically calculate the distance from the coordinates of the pair of measuring electrodes. Herein the course of a coronary artery may be determined from its origin by selecting the neigbouring pair of measuring electrodes to give the strongest signal.

The display means 16 may have one screen, on which one coronary artery at a time may be shown, but is also possible to have a screen on which every one of the nine major surface coronary arteries are shown.

Furthermore it is possible to connect the processing means 14 to an optional heart display means 16. This is a graphical representation of the surface coronary vasculature of a human heart. The measured peak blood flow signals may be indicated on this graphical representation of the heart. This way it is e.g. possible to show the location of special points of interest, such as stenosis, i.e. locations where the artery is narrowed or constricted.

The device may be used to determine a blood flow signal in one or more coronary arteries, with the help of a method according to the invention.

In the method, impedance signals are measured across the heart 5 or parts thereof. Thereto, a current field is established across the heart 5, or at least across a part of the heart, by means of the current source 1 and the supply electrodes 2a,b. The current produces a voltage drop across any impedance. Every piece of tissue forms an impedance. Changes in the impedance are measured as a change in the voltage. The most important impedance change is due to the flow of blood caused by the heart beat.

It has been found, by the inventor, that the blood flow through the coronary arteries, particularly the surface coronary arteries, is related to two small details in the impedance signal. During the isovolumetric relaxation period of the heart beat, that is during early diastole, no blood enters or leaves the heart. However, the left and right coronary arteries rapidly fill with blood. Since the left coronary artery, which is in fact a system of coronary arteries, has a larger volume and a lower system resistance than the right coronary arteries (system), the signal caused by the left coronary arteries system precedes that of the right coronary arteries system. Hence these signals may be processed separately. This will be further elucidated in connection with FIGS. 2a-d and 3.

FIGS. 2a and 2b show an example of a measured impedance signal Z, the first time-derivative dZ/dt thereof, respectively, as measured across a complete heart, i.e. not just a part of a selected coronary artery. This is done to be better able to explain the processes that occur during a heart beat. FIG. 2c shows a phonocardiogram, i.e. a diagram of the measured heartsounds. FIG. 2d shows a well known EKG or electrocardigram.

It is to be noted here that in reality the measured signals Z and dZ/dt are inverted. It is only out of custom that the large peak is a positive one in the drawing. There is however no influence on the values of the peak blood flow signal, since only absolute values are to be used.

In FIGS. 2a to d three heart beats are shown. In FIG. 2a three large peak signals 20 are visible, basically due to the pumping of blood into the aorta. This pumping action is even more visible in FIG. 2b, where it is visible as three pronounced peaks 21. Each “aorta pumping peak” 21 is followed by a somewhat less pronounced negative peak 22. This negative peak 22 represents the closing of the aortic valve, and also the end of systole 25 and the beginning of diastole 26. This latter phase of relaxing of the heart begins with the isovolumetric relaxation time (IVRT) 27. In this IVRT 27, all heart valves are closed, hence no blood enters or leaves the heart. The heart muscle relaxes. The IVRT ends with the opening of the mitral valve, which is represented by beginning of the O-wave 28. Between the closing of the aortic valve and the opening of the mitral valve two more peaks 29, 30 are visible.

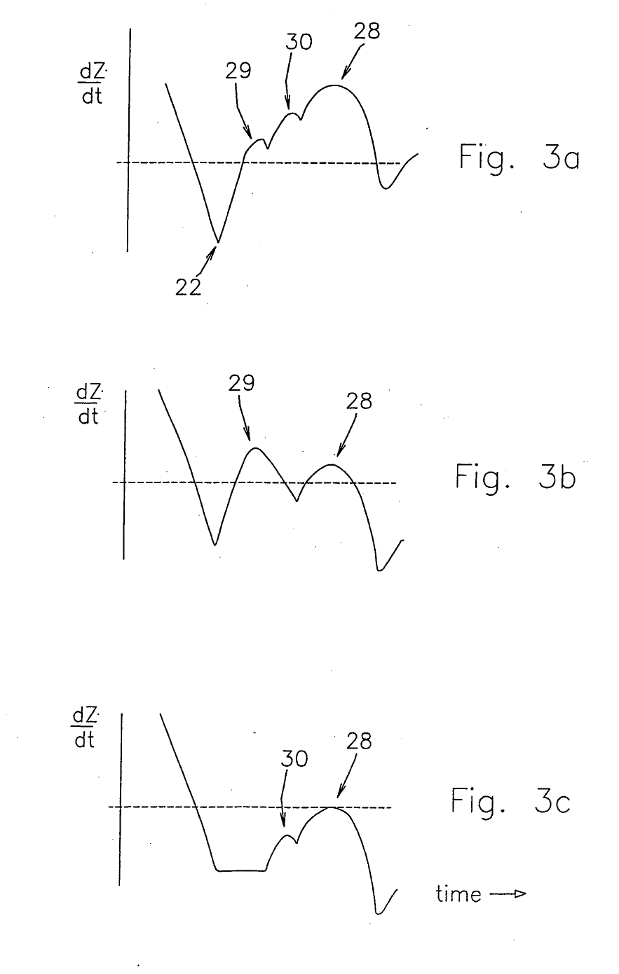

It was shown in experiments that these peaks 29, 30 could be made to disappear when the coronary arteries where blocked, e.g. by means of an inflatable balloon, the first peak signal 29 when the left coronary artery was blocked, and the second peak signal 30 when the right coronary artery was blocked. This is shown in FIG. 3a to c. which is an enlargement of the indicated detail, and showing the effects of closing off left or right coronary arteries.

FIG. 3a shows a complete dZ/dt signal, with no coronary arteries blocked. FIG. 3b shows a signal in which the right coronary artery is blocked, leaving only the left coronary peak signal 29. And FIG. 3c shows a signal in which the left coronary artery is blocked, leaving only the right coronary peak signal 30. Note that in each case the O-wave 28 is shifted to a lower level, because of the absence of either left or right coronary filling. Furthermore, in FIG. 3c may be seen that in the absence of left coronary filling, i.e. no peak 29, there is a flat part in the dZ/dt signal. This indicates that no blood at all is moved through the heart, neither through the ventricles, atria, aorta or coronary arteries.

Thus, it is possible to separate the coronary artery peak signals. First of all, when not the complete heart 5 is measured, but only a small part thereof, by applying a mesh 4 of electrodes and selecting a part of a coronary artery bounded between two chosen measuring electrodes, it turns out that the measured signal is only contributed to by that bounded part of that selected coronary artery. The other coronary arteries do not influence the signal. It is thought to be caused by the fact that signals from coronary arteries on one side of the heart are separated from signals from coronary arteries on the opposite side of the heart, because of the intermediate heart tissue and cardiac chambers. This dampens the signals in the direction of their “opposite neighbours”. Basically, the signals from the coronary arteries may be treated as surface signals, when measured with a fine mesh. As a result, it is possible to select a coronary artery signal 29, 30 as the first peak signal to occur after the negative peak 22 that corresponds to the closing of the aortic valve. It is to be noted that, by thus separating the two peak signals that correspond to the left coronary artery system, the right coronary artery system, respectively, the peak signals themselves become more pronounced, as will be seen in FIG. 3, to be discussed here below.

Furthermore, it is possible, though not necessary, to filter out the coronary peak signal (or signals, when for some reason both coronary peak signals are measured at the same time) from the total dZ/dt-signal by a gating procedure. The gate opens at the time of closing of the aortic valve, and is allowed to close at or somewhat after the opening of the mitral valve, since at that point in time the peak signal is added to the signal due to the ventrical filling (O-wave 28), and hence less pronounced. Since, however, it is the height of the coronary peaks 29, 30 which is important, this does not influence the accuracy of the processed signals.

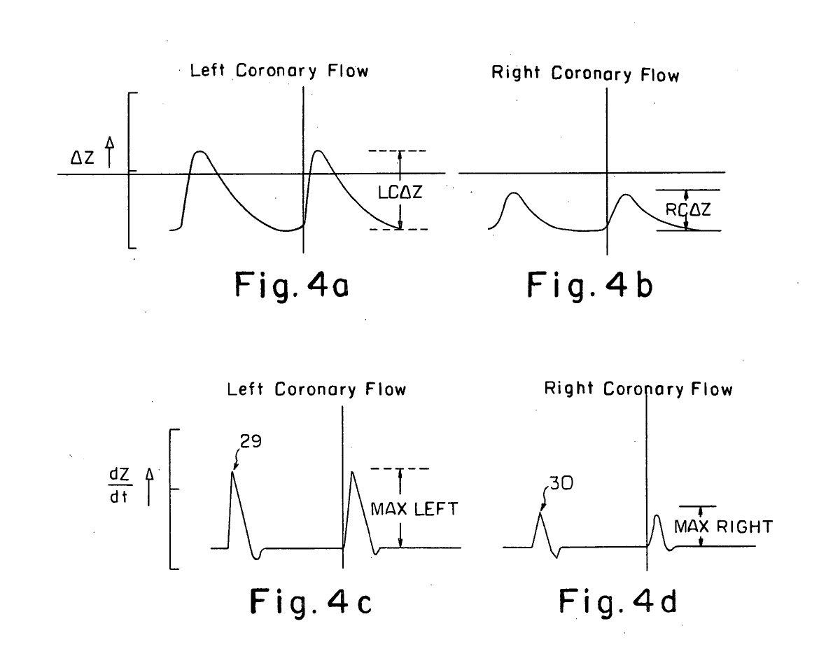

FIGS. 4a,b show separated Z-signals. These may be measured either directly, or through integrating separated dZ/dt signals. However, they are not used in the present method. FIGS. 4c,d show first time-derivates dZ/dt of the separated Z-signals, with indicated maximum value MAX left, MAX right, for the left, the right coronary artery, respectively. These signals are analogue signals, taken at a representative point of the coronary arteries. It could also be a digitized version of the signal. In fact, signals 4c,d represent the peak signals PS which are processed in the present method.

From the signals PS 29 and 30 of FIGS. 4c and d, the corresponding peak blood flow signal PF may be determined by dividing the value of MAX for that signal by the inter-electrode distance. If a constant inter-electrode distance is taken, which is preferred, this step may be omitted.

The peak blood flow signal PF thus found, corresponds to a specific location along the selected coronary artery. When in this way the peak blood flow signal is determined for many points along the selected coronary artery, a profile of the blood flow through that artery may be obtained. Examples of such blood flow profile may be seen in FIG. 1, in the display means 16.

Herein, all nine major surface coronary arteries are shown, together with their measured peak blood flow signal profile. Generally, a slightly rising peak profile should be seen. This is caused by the slight rise in blood flow velocity due to a decrease in the cross sectional area of a coronary artery in the direction of blood flow. This generally increasing peak blood flow signal may be seen in the arteries LM, LPD, RC, RM and RPD, wherein common abbreviations for their names have been used.

Some other details are visible. For example, in the LAD, a peak in the profile is visible. This indicates a local narrowing or stenosis of the LAD artery, which narrowing causes a local increase in the peak blood flow velocity. Furthermore, in the RCA, a similar peak is visible, which is however followed by a zero signal. This indicates a stenosis which completely occludes the RCA artery, since a zero signal corresponds to zero blood flow.

The information about the coronary arteries thus obtained may be indicated in the heart display means 16. The location of stenosis and/or complete occlusion may be highlighted with a different intensity or color of light, etcetera. The doctor may use this graph to explain the situation to the patient, a colleague, or a surgeon who is to perform surgery.

Various other modifications of the disclosed embodiments of the invention will become apparent to persons skilled in the art upon reference to the description and drawing. It is therefore contemplated that the appended claims will cover such modifications or embodiments as fall within the true scope of the invention.

1. A device for determining a peak blood flow signal of a blood flow through at least a section of a selected coronary artery of a beating heart of a mammal, in particular a human being, wherein said device comprises a bioimpedance measuring device, which at least comprises
at least two measuring electrodes,
an impedance measuring device for measuring an impedance signal Z between a pair of said measuring electrodes, and
processing means, which are able to determine a first time-derivative dZ/dt for said measured impedance signal Z, and to separate from said first time-derivative dZ/dt a peak signal PS that occurs first after the beginning of diastole of said heart during a heart beat,
and wherein said processing means are able to determine a maximum value MAX of said peak signal PS.

2. A device according to claim 1, wherein said bioimpedance measuring device comprises a plurality of measuring electrodes.

3. A device according to claim 2, wherein said plurality of measuring electrodes is arranged in a mesh.

4. A device according to claim 3, wherein all neighbouring measuring electrodes are substantially equidistant.

5. Device according to claim 1, wherein the processing means are able to determine a peak blood flow signal PF for said peak signal PS, by dividing said maximum value MAX by the distance between those two of said at least two measuring electrodes between which the impedance signal Z was measured, when the device is being used.

6. A device according to claim 2, wherein said processing means further comprise scanning means, which are able to select two or more pairs of said plurality of measuring electrodes during said heart beat, the processing means for each of said pairs being able to determine said peak blood flow signal PF in that section of said selected coronary artery which is bounded by said pair of measuring electrodes selected by the scanning means.

7. A device according to claim 6, wherein said scanning means are able to select all pairs of neighbouring measuring electrodes during one heart beat.

8. A device according to claim 1, further comprising display means for representing said peak blood flow signal PF as a function of position along said at least one selected coronary artery.

9. A device according to claim 2, wherein said plurality of measuring electrodes can substantially cover said heart.

10. A method for determining a peak blood flow signal PF of a blood flow through at least a section of at least one selected coronary artery of a beating heart of a mammal, in particular a human being, comprising the steps of
applying a bioimpedance measuring device at least comprising impedance measuring means and at least two mutually spaced measuring electrodes to the body of said mammal, wherein at least said section of said at least one selected coronary artery is bounded by a pair of measuring electrodes of said at least two measuring electrodes,
measuring an impedance signal Z between said pair of measuring electrodes, by means of said bioimpedance measuring device, which signal Z depends on the blood flow through that section of said at least one selected coronary artery which is bounded by said pair of measuring electrodes,
determining a first time-derivative dZ/dt of said impedance signal
separating from said first time-derivative dZ/dt a peak signal PS that occurs first after the beginning of diastole of said heart during a heart beat,
determining for said peak signal PS a maximum value MAX of said peak signal PS.

11. A method according to claim 10, wherein a plurality of impedance signals Z are determined along a plurality of locations along said at least one coronary artery, wherein for each of said impedance signals Z a first time-derivative dZ/dt is determined, a peak signal PS is separated therefrom, and a maximum value MAX is determined from said peak signal PS.

12. A method according to claim 10, wherein a peak blood flow signal PF is determined for said peak signal PS by dividing said maximum value MAX of said peak signal PS by the distance between those electrodes between which said peak signal PS was determined.

13. A method according to claim 10, wherein a plurality of measuring electrodes is applied to said body.

14. A method according to claim 13, further comprising the use of scanning means which are able to select at least two pairs of measuring electrodes from said plurality of measuring electrodes.

15. A method according to claim 12, further comprising the step of representing said peak blood flow signal PF as a function of position along said at least one selected coronary artery.

16. A method according to claim 15, further comprising the step of graphically highlighting those sections along said at least one selected coronary artery in which an increase in said peak blood flow signal PF is followed by a decrease in said peak blood flow signal PF, as seen in the direction of said blood flow.

17. A method according to claim 16, wherein said graphically highlighted parts are indicated on a model of a surface vasculature of said heart.

18. A method according to claim 10, wherein substantially all major surface coronary arteries of said heart are selected.

19. A method according to claim 13, wherein said plurality of measuring electrodes covers said heart substantially completely.
